(19) United States
(12) Patent Application Publication
(10) Pub. No.: US 2014/0296536 A1
(43) Pub. Date: Oct. 2, 2014

(54) METHOD FOR PRODUCING DITHIINE TETRACARBOXIMIDES

(71) Applicant: Bayer Intellectual Prooperty GmbH, Monheim (DE)

(72) Inventors: Thomas Himmler, Odenthal (DE); Thomas Geller, Odenthal (DE); Lars Rodfefeld, Leverkusen (DE); Frank Volz, Koeln (DE)

(21) Appl. No.: 14/350,631

(22) PCT Filed: Oct. 11, 2012
(86) PCT No.: PCT/EP2012/070105

§ 371 Date: Apr. 9, 2014

(30) Foreign Application Priority Data
Oct. 13, 2011 (EP) 11185002.0

(51) Int. Cl. C07D 495/14

(52) U.S. Cl. CPC C07D 495/14
USPC 548/431

(57) ABSTRACT

The present invention relates to a new process for preparing dithiine-tetracarboximides.

The present invention relates to a new process for preparing dithiine-tetracarboximides.

Dithiine-tetracarboximides as such are already known. It is also known that these dithiine-tetracarboximides can be used as anthelmintics against internal parasites of animals, more particularly nematodes, and have insecticidal activity (cf. U.S. Pat. No. 3,364,229). It is known, furthermore, that certain dithiine-tetracarboximides possess antibacterial activity and have a certain activity against causative organisms of human mycoses (cf. Il Farmaco 2005, 60, 944-947). It is also known that dithiine-tetracarboximides can be used as pigments in electrophotographic photoreceptors or as dyes in paints and polymers (cf. JP-A 10-251265, PL-B 143804).

Dithiine-tetracarboximides of the formula (I)

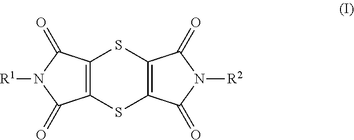

in which
R¹ and R² are identical or different and are hydrogen, or are C₁-C₈-alkyl which is optionally substituted one or more times by halogen, —OR³, and/or —COR⁴, are C₃-C₇-cycloalkyl which is optionally substituted one or more times by halogen, C₁-C₄-alkyl or C₁-C₄-haloalkyl, or are aryl or aryl-(C₁-C₄-alkyl) each of which is optionally substituted one or more times by halogen, C₁-C₄-alkyl, C₁-C₄-haloalkyl, —COR⁴ or sulphonylamino,
R³ is hydrogen, C₁-C₄-alkyl or C₁-C₄-alkylcarbonyl or is aryl which is optionally substituted one or more times by halogen, C₁-C₄-alkyl or C₁-C₄-haloalkyl,
R⁴ is hydroxyl, C₁-C₄-alkyl or C₁-C₄-alkoxy,
can be prepared in a variety of known ways.

For example, in one process (cf. U.S. Pat. No. 3,364,229; Chem. Ber. 1967, 100, 1559-1570), in a first stage, dichloromaleic anhydride of the formula (II) is reacted with an amine of the formula (III), optionally in the presence of a diluent. Subsequently, the resultant dichloromaleimides of the formula (IV) are then reacted with a sulphur compound (e.g. hydrogen sulphide or thiourea). The preparation of the dithiine-tetracarboximides of the formula (I) by this process can be illustrated by the following scheme:

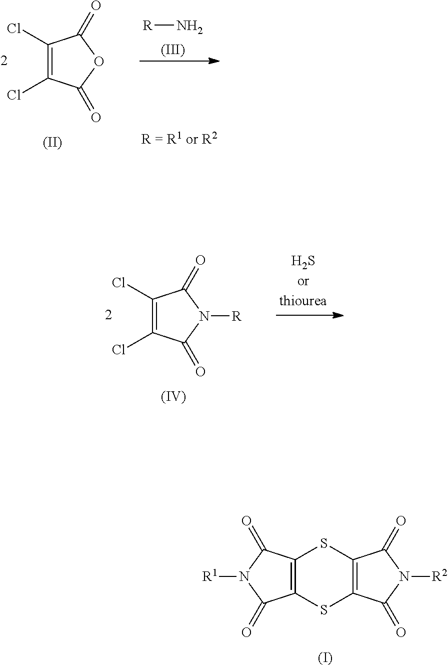

This process has the disadvantage that, for example, operating with the highly toxic gaseous hydrogen sulphide is from a technical standpoint very difficult, costly and inconvenient. When thiourea is used, unwanted by-products are obtained along with the target product, and are very difficult to remove, and detract from the attainable yields (cf. J. Heterocycl. Chem. 1988, 25, 901-906).

In another process which has been disclosed (cf. Synthetic Communications 2006, 36, 3591-3597), in a first stage, succinic anhydride of the formula (V) is reacted with an amine of the formula (III), optionally in the presence of a diluent. Subsequently, the resultant succinic monoamides of the formula (VI) are reacted for 6 hours with a large excess of thionyl chloride in the presence of dioxane as diluent, at room temperature, to give, finally, in a sequence of numerous reaction steps, the dithiine-tetracarboximides of the formula (I). The dithiine-tetracarboximides are optionally isolated directly from the reaction mixture or by filtration following addition of water. Depending on reaction conditions (diluents) and the nature of the radicals R, it is possible in certain circumstances to isolate the dithiine-diisoimides of the formula (VII) before they are converted into the dithiine-tetracarboximides of the formula (I). This preparation method for the dithiine-tetracarboximides of the formula (I) can be illustrated by the following scheme:

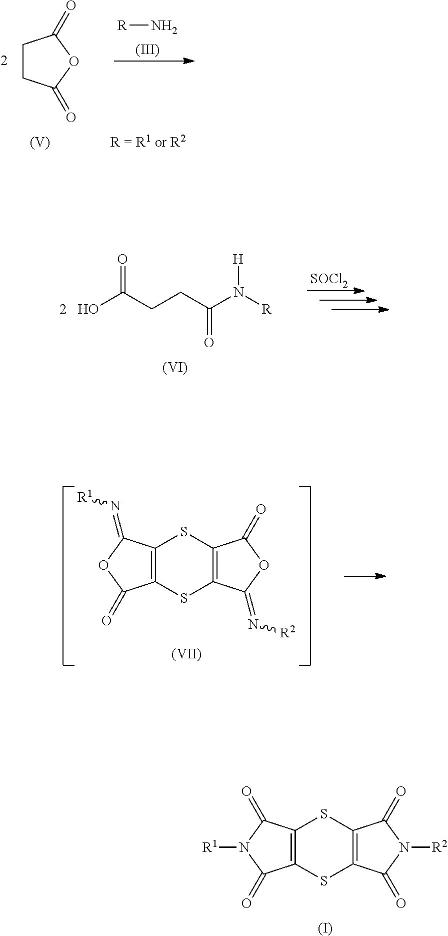

Disadvantages of this process are the long reaction time and also the outcome where either the yields obtained generally do not exceed about 30-40% of theory or else the purities of the isolated products are inadequate (see comparative examples). A further disadvantage, in the case of aqueous work-up of the reaction mixture, is that it involves destroying large amounts of thionyl chloride; the gases formed (SO₂ and HCl) have to be disposed of. Likewise a disadvantage is the fact that, from experience (see comparative examples), the product is not obtained in one fraction. Instead, it is frequently the case that, following initial isolation of product by filtration, further product precipitates from the filtrate after prolonged standing (overnight, for example), and must be isolated again by filtration. Occasionally this operation must be carried out once more. This procedure is very laborious and time-consuming.

Consequently there continues to be a need for a technically simple and economic preparation process for dithiine-tetracarboximides of the formula (I).

A new process has been found for preparing dithiine-tetracarboximides of the general formula (I)

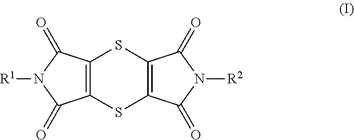

in which R¹ and R² have the definitions indicated above,
characterized in that
in a first stage, succinic monoamide carboxylates of the formula (VI)

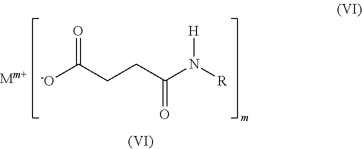

in which R is R¹ or R²,
M is a cation selected from the group consisting of alkali metals, alkaline earth metals, transition metals and metals and
m is 1, 2, 3 or 4,
are reacted with an excess of thionyl chloride, optionally in the presence of a diluent,
then the excess of thionyl chloride is removed and the resulting product mixture is converted in a second stage, in a mixture of an organic solvent, water and a phase transfer catalyst into the dithiine-tetracarboximides of the formula (I).

In this way the dithiine-tetracarboximides of the formula (I) can be obtained in relatively high yield, a relatively short time, and relatively good purity. It is, moreover, possible to recover the organic solvent.

The product mixture obtained in the first step of the process of the invention also already includes dithiine-tetracarboximides of the formula (I), but its principal components are polysulphides of the formula (IX),

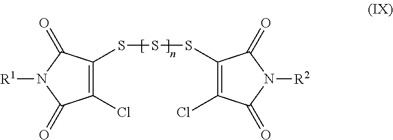

and also, depending on the work-up method, thiosulphonic acid derivatives of the formula (VIII)

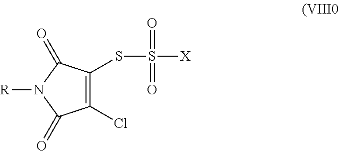

In the thiosulphonic acid derivatives of the general formula (VIII), R stands for the definitions of R¹ and R², indicated above, and X stands for chlorine or hydroxyl.

In the polysulphides of the general formula (IX), R¹ and R² stand for the definitions indicated above, and n stands for 0, 1, 2, 3, 4, 5, 6, 7 or 8.

Compounds of the general formula (VIII) are obtained, alongside other products, when the reaction mixture, following the reaction of the compounds of the general formula (VI) with thionyl chloride, is concentrated.

Compounds of the general formula (IX) are obtained, alongside other products, when the reaction mixture, following the reaction of the compounds of the general formula (VI) with thionyl chloride, is concentrated, dissolved in an inert, water-immiscible solvent such as methylene chloride, for example, and extracted by shaking with water at room temperature. Following removal of the organic phase, drying and concentrating, a mixture is obtained which in addition to dithiine-tetracarboximides of the formula (I) contains primarily compounds of the general formula (IX).

The process of the invention for preparing the dithiine-tetracarboximides of the formula (I) can be illustrated by the following scheme:

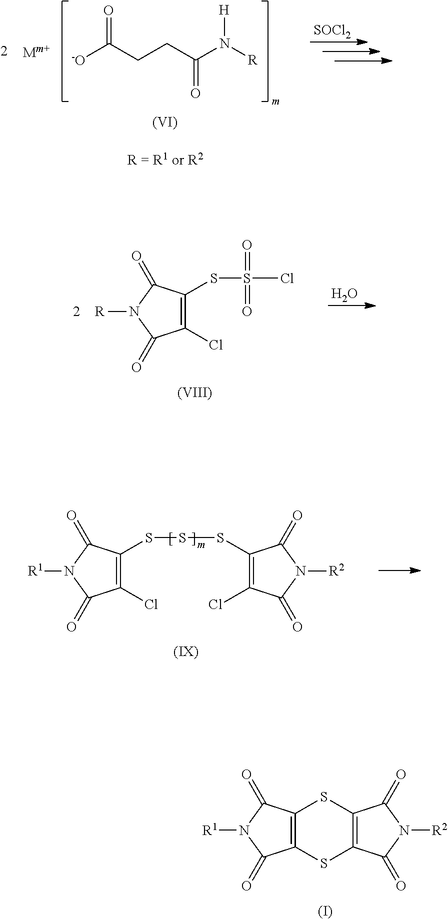

A general definition of the succinic monoamide carboxylates used as starting materials when carrying out the process of the invention is provided by the formula (VI). R stands for the definitions of R¹ or R².

R¹ and R² preferably are identical or different and preferably are hydrogen, or are C₁-C₆-alkyl which is optionally substituted one or more times by fluorine, chlorine, bromine, —OR³ and/or —COR⁴, or are C₃-C₇-cycloalkyl which is optionally substituted one or more times by chlorine, methyl or trifluoromethyl, or are phenyl or phenyl-(C₁-C₄-alkyl) each of which is optionally substituted one or more times by fluorine, chlorine, bromine, methyl, trifluoromethyl, —COR⁴ and/or sulphonylamino.

R¹ and R² more preferably are identical or different and more preferably are hydrogen, or are C₁-C₄-alkyl which is optionally substituted one or more times by fluorine, chlorine, hydroxyl, methoxy, ethoxy, methylcarbonyloxy and/or carboxyl, or are C₃-C₇-cycloalkyl which is optionally substituted one or more times by chlorine, methyl or trifluoromethyl, or are phenyl, benzyl, 1-phenethyl, 2-phenethyl or 2-methyl-2-phenethyl each of which is optionally substituted one to three times by fluorine, chlorine, bromine, methyl, trifluoromethyl, —COR⁴ and/or sulphonylamino.

R¹ and R² very preferably are identical or different and very preferably are hydrogen, methyl, ethyl, n-propyl, isopropyl, 2,2-difluoroethyl or 2,2,2-trifluoroethyl or are cyclopropyl or cyclohexyl each of which is optionally substituted by chlorine, methyl or trifluoromethyl.

R¹ and R² more sarticularl referabl are simultaneously methyl.

R³ preferably is hydrogen, methyl, ethyl, methylcarbonyl or ethylcarbonyl or is phenyl which is optionally substituted one or more times by fluorine, chlorine, methyl, ethyl, n-propyl, isopropyl or trifluoromethyl.

R³ more preferably is hydrogen, methyl, methylcarbonyl or phenyl.

R⁴ preferably is hydroxyl, methyl, ethyl, methoxy or ethoxy.

R⁴ more preferably is hydroxyl or methoxy.

M preferably is Li, Na, K, Rb, Cs with

m as 1,
or
Be, Mg, Ca, Sr, Ba, with

m as 2,

or
Ti, V, Mn, Fe, Co, Ni, Cu, Zn, Al with

m as 1, 2, 3 or 4.

M more preferably is Li, Na, K, with

m as 1,

or
Be, Mg, Ca, with

m as 2,

or
Mn, Fe, Co, Al with

m as 1, 2, 3 or 4.

M very preferably is Na, K, with

m as 1,

or
Mg, Ca, with

m as 2,

or
Mn, Fe, Al with

m as 2, 3 or 4.

As starting material it is particularly preferred to use N-methylsuccinamide carboxylates, giving as the end product the compound (I-1) 2,6-dimethyl-1H,5H-[1,4]dithiino[2,3-c:5,6-c′]dipyrrole-1,3,5,7(2H,6H)-tetrone.

If sodium N-tert-butylsuccinamide carboxylate is used as starting material, the end product obtained is the compound (I-2) 2,6-di-tert-butyl-1H,5H-[1,4]dithiino[2,3-c:5,6-c′]dipyrrole-1,3,5,7(2H,6H)-tetrone.

If sodium N-cyclohexylsuccinamide carboxylate is used as starting material, the end product obtained is the compound (I-3) 2,6-dicyclohexyl-1H,5H-[1,4]dithiino[2,3-c:5,6-c′]dipyrrole-1,3,5,7(2H,6H)-tetrone.

If sodium N-propylsuccinamide carboxylate is used as starting material, the end product obtained is the compound (I-4) 2,6-dipropyl-1H,5H-[1,4]dithiino[2,3-c:5,6-c′]dipyrrole-1,3,5,7(2H,6H)-tetrone.

Intermediates obtained with particular preference are

(VIII-1) S-(4-chloro-1-methyl-2,5-dioxo-2,5-dihydro-1H-pyrrol-3-yl) chlorothiosulphate (R=Me, X=Cl),

(IX-1) 3,3′-trisulphane-1,3-diylbis(4-chloro-1-methyl-1H-pyrrole-2,5-dione) (R¹=R²=Me, n=1)

(IX-2) 3,3′-disulphanediylbis(4-chloro-1-methyl-1H-pyrrole-2,5-dione) (R¹=R²=Me, n=0)

(IX-3) 3,3′-disulphanediylbis(1-tert-butyl-4-chloro-1H-pyrrole-2,5-dione) (R¹=R²=t-Bu, n=0)

(IX-4) 3,3′-trisulphane-1,3-diylbis(1-tert-butyl-4-chloro-1H-pyrrole-2,5-dione) (R¹=R²=t-Bu, n=1)

(IX-5) 3,3′-trisulphane-1,3-diylbis(4-chloro-1-cyclohexyl-1H-pyrrole-2,5-dione) (R¹=R²=cyclohexyl, n=1)

The amount of thionyl chloride in the first step of the process of the invention is between 2 and 100 mol per mole of succinic monoamide carboxylate of the formula (VI). It is preferred to use between 2 and 50 mol, more preferably amounts of between 4 and 40 mol, per mole of succinic monoamide carboxylate of the formula (VI).

The reaction temperature in the first step of the process of the invention can be varied within wide limits and is between 0° C. and 150° C. In order to obtain satisfactory space-time yields, it is preferred to operate at temperatures between 20° C. and 120° C., more preferably between 30° C. and 100° C.

The reaction time in the first step of the process of the invention is between 10 minutes and 24 hours. It is preferred to operate for between 30 minutes and 6 hours, more preferably between 1 and 4 hours.

The first step of the process of the invention can be carried out optionally in the presence of a diluent which as far as possible is inert under the reaction conditions. Such diluents include, by way of example, aliphatic hydrocarbons such as pentane, hexane, heptane, cyclohexane, methylcyclohexane, octane, isooctane, chlorinated hydrocarbons such as methylene chloride, chloroform, 1,2-dichloroethane, aromatic hydrocarbons such as toluene, xylene, mesitylene, ethylbenzene, anisol, chlorinated aromatic hydrocarbons such as chlorobenzene, dichlorobenzene, ethers such as diethyl ether, methyl tert-butyl ether, tetrahydrofuran, dioxane, nitriles such as acetonitrile, propionitrile, butyronitrile, esters such as methyl acetate and ethyl acetate. It is preferred to operate in toluene, xylene, mesitylene, ethylbenzene, chlorobenzene or 1,2-dichlorobenzene or without diluent.

The thionyl chloride can be removed in principle by hydrolysis with water. The thionyl chloride is removed preferably by distillation under reduced pressure.

The diluent optionally present may likewise be distilled off under reduced pressure and, if desired, be replaced by another solvent. Preferably, however, only the excess thionyl chloride is distilled off and then, after addition of water and phase transfer catalyst, the reaction is continued in the same solvent.

In the second step of the process of the invention, the residue that is obtained following removal of the excess thionyl chloride and optionally of the diluent is dissolved in a new diluent and, after addition of a phase transfer catalyst, by heating in this solvent, is converted into the dithiine-carboximides of the formula (I). The reaction mixture is preferably stirred during this procedure.

In the second step of the process of the invention, organic solvents or solvent mixtures are used. These solvents are preferably only slightly miscible with water.

Suitable diluents for the second step of the process of the invention include, specifically, hydrocarbons such as hexane, heptane, cyclohexane, methylcyclohexane, octane, isooctane, toluene, xylenes, mesitylene, ethylbenzene, chlorobenzene, dichlorobenzene, nitrobenzene, water or mixtures of these diluents.

Preference is given to using hexane, heptane, cyclohexane, methylcyclohexane, octane, isooctane, toluene, xylenes, mesitylene, chlorobenzene, dichlorobenzene, water or mixtures of these diluents.

Very particular preference is given to using mixtures of water and toluene, xylene or chlorobenzene.

The mixing ratio of water to organic solvent here may be varied within wide limits of, for example, 9:1 to 1:9.

As phase transfer catalysts (PTC) it is possible in principle to use all compounds with a known activity as PTC. Such compounds may be, for example, phase transfer catalysts from the series of the quaternary ammonium salts or of the quaternary phosphonium salts.

This phase transfer catalyst preferably possesses the general formula (X)

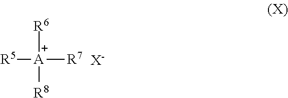

in which
R⁵, R⁶, R⁷ and R⁸ independently of one another are identical or different and are each straight-chain or branched C₁-C₂₈-alkyl, C₆-C₁₀-aryl or benzyl,
X is halogen, hydrogen sulphate, sulphate, dihydrogen phosphate, hydrogen phosphate, phosphate or acetate (preferably bromine, chlorine, fluorine, hydrogen sulphate, sulphate, phosphate and acetate),

### A is N or P.

Examples that may be given of such phase transfer catalysts include tetrabutylammonium fluoride, chloride, bromide, iodide, acetate and hydrogen sulphate, tetraethylammonium bromide and iodide, methyltributylammonium chloride, bromide, iodide, acetate and hydrogen sulphate, benzyldodecyldimethylammonium chloride and bromide, benzyltriethylammonium bromide and chloride, dodecyltrimethylammonium chloride and bromide, tetradecyltrimethylammonium chloride and bromide, methyltrioctylammonium chloride, methyltridecylammonium chloride, tetraoctylammonium bromide and chloride, didecyldimethylammonium chloride and bromide, tetraphenylphosphonium bromide, ethyltriphenylphosphonium bromide, ethyltriphenylphosphonium iodide and ethyltriphenylphosphonium acetate.

In addition it is also possible for phase transfer catalysts such as 4-dialkylaminopyridinium salts or hexaalkylguanidinium salts to be employed.

Preference is given to using methyltrioctylammonium chloride (trade name Aliquat® 336; present in a mixture with methyltridecylammonium chloride), methyltridecylammonium chloride or bromide, tetraoctylammonium bromide or chloride, dodecyltrimethylammonium chloride or bromide, tetradecyltrimethylammonium chloride or bromide, didecyldimethylammonium chloride or bromide and benzyldodecyldimethylammonium chloride or bromide for use as phase transfer catalysts.

The amount of phase transfer catalyst in the process of the invention can be varied within wide limits. The amount is preferably between 0.1 and 10 mol percent, based on the succinimide carboxylate of the formula (VI), more preferably between 1 and 7 mol percent, based on the succinimide carboxylate of the formula (VI).

In a further embodiment of the process it is possible for the filtrate which is obtained after the isolation of the product of the general formula (I) by filtration and which is two-phase when—as preferred—a mixture is used of water and an organic diluent which is immiscible or only a little miscible with water, to be employed in the next batch for carrying out the second step of the process of the invention. This can be repeated a number of times, preferably up to ten times, more preferably up to five times. As a result, not only is the use of the phase transfer catalyst significantly reduced, relative to succinimide carboxylate of the general formula (VI), but also, at the same time, the required amounts of organic diluent and water are significantly reduced, and this makes the process even more economical.

Depending on which phase transfer catalyst is used, it is also possible for only the organic phase, after removal of the desired product, to be re-used.

The reaction temperature in the second step of the process of the invention can be varied within wide limits and is between 0° C. and 200° C. It is preferred to operate at temperatures between 20° C. and 150° C., more preferably between 30° C. and 130° C.

The reaction time in the second step of the process of the invention is between 5 minutes and 24 hours. It is preferred to operate for between 30 minutes and 12 hours, more preferably between 1 and 8 hours.

1. Process for preparing a dithiine-tetracarboximide of formula (I) 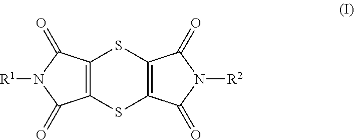
in which
R¹ and R² are identical or different and are hydrogen, or are C₁-C₈-alkyl which is optionally substituted one or more times by halogen, —OR³, and/or —COR⁴, are C₃-C₇-cycloalkyl which is optionally substituted one or more times by halogen, C₁-C₄-alkyl or C₁-C₄-haloalkyl, or are aryl or aryl-(C₁-C₄-alkyl) each of which is optionally substituted one or more times by halogen, C₁-C₄-alkyl, C₁-C₄-haloalkyl, —COR⁴ or sulphonylamino,
R³ is hydrogen, C₁-C₄-alkyl or C₁-C₄-alkylcarbonyl or is aryl which is optionally substituted one or more times by halogen, C₁-C₄-alkyl or C₁-C₄-haloalkyl,
R⁴ is hydroxyl, C₁-C₄-alkyl or C₁-C₄-alkoxy,
comprising reacting
in a first stage, a succinic monoamide carboxylate of formula (VI) 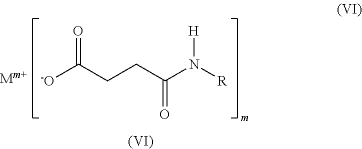
in which R is R¹ or R²
and
M is a cation selected from the group consisting of alkali metals, alkaline earth metals, transition metals and metals and
m is 1, 2, 3 or 4,
with an excess of thionyl chloride, optionally in the presence of a diluent,
then excess of thionyl chloride is removed and resulting product mixture is converted in a second stage, in a mixture of an organic solvent, water and a phase transfer catalyst into the dithiine-tetracarboximide of formula (I).

2. Process according to claim 1, wherein in the second stage, the phase transfer catalyst is at least one selected from
(a) quaternary ammonium salts or quaternary phosphonium salts of formula (X) 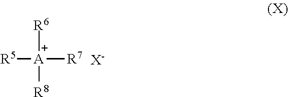
in which
R⁵, R⁶, R⁷ and R⁸ independently of one another are identical or different and are each straight-chain or branched C₁-C₂₈-alkyl, C₆-C₁₀-aryl or benzyl,
X is halogen, hydrogen sulphate, sulphate, dihydrogen phosphate, hydrogen phosphate, phosphate or acetate optionally (bromine, chlorine, fluorine, hydrogen sulphate, sulphate, phosphate and/or acetate,
A is N or P;
or
(b) 4-dialkylaminopyridinium salts or hexaalkylguanidinium salts.

3. Process according to claim 1, wherein in the first stage a succinic monoamide carboxylate of formula (VI) 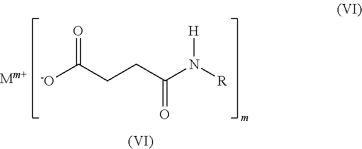
in which R is R¹ or R²,
and
M is Li, Na, K, Rb, Cs with
m as 1,
or
Be, Mg, Ca, Sr, Ba, with
m as 2,
or
Ti, V, Mn, Fe, Co, Ni, Cu, Zn, Al with
m as 1, 2, 3 or 4,
is reacted with an excess of thionyl chloride, optionally in the presence of a diluent, and then excess of thionyl chloride is removed and resulting product mixture is converted in a second stage, in a mixture of an organic solvent, water and a phase transfer catalyst, into the dithiine-tetracarboximide of formula (I).

4. Process according to claim 1, wherein in the first stage a succinic monoamide carboxylate of formula (VI) 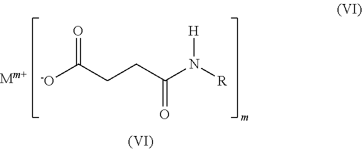
in which R is R¹ or R²,
and
M is Li, Na, K, with
m as 1,
or
Be, Mg, Ca, with
m as 2,
or
Mn, Fe, Co, Al with
m as 2, 3 or 4,
is reacted with an excess of thionyl chloride, optionally in the presence of a diluent, and then excess of thionyl chloride is removed and resulting product mixture is converted in a second stage, in a mixture of an organic solvent, water and a phase transfer catalyst, into the dithiine-tetracarboximide of formula (I).

5. Process according to claim 1, wherein in the first stage succinic monoamide carboxylate of formula (VI) 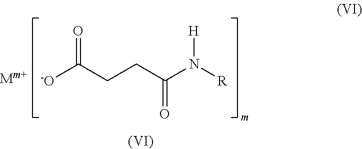
in which R is R¹ or R²,
and
M is Na, K, with
m as 1,
or
Mg, Ca, with
m as 2,
or
Mn, Fe, Al with
m as 2, 3 or 4,
is reacted with an excess of thionyl chloride, optionally in presence of a diluent, and then excess of thionyl chloride is removed and resulting product mixture is converted in a second stage, in a mixture of an organic solvent, water and a phase transfer catalyst, into the dithiine-tetracarboximide of formula (I).

6. Process according to claim 1, wherein in the second stage a phase transfer catalyst from the following list is selected: tetrabutylammonium fluoride, chloride, bromide, iodide, acetate and hydrogen sulphate, tetraethylammonium bromide and iodide, methyltributylammonium chloride, bromide, iodide, acetate and hydrogen sulphate, benzyldodecyldimethylammonium chloride and bromide, benzyltriethylammonium bromide and chloride, dodecyltrimethylammonium chloride and bromide, tetradecyltrimethylammonium chloride and bromide, methyltrioctylammonium chloride, methyltridecylammonium chloride, tetraoctylammonium bromide and chloride, didecyldimethylammonium chloride and bromide, tetraphenylphosphonium bromide, ethyltriphenylphosphonium bromide, ethyltriphenyl-phosphonium iodide and ethyltriphenylphosphonium acetate.

7. Process according to claim 1, wherein in the stage an organic solvent is used which is only slightly miscible with water.

8. Process according to claim 1, wherein hexane, heptane, cyclohexane, methylcyclohexane, octane, isooctane, toluene, xylenes, mesitylene, ethylbenzene, chlorobenzene, dichlorobenzene, nitrobenzene, water or mixtures of these diluents are used in the second stage.

9. Process according to claim 1, wherein filtrate which is obtained after isolation of product of formula (I) by filtration, and which is two-phase when a mixture is used of water and an organic diluent which is immiscible or only a little miscible with water, is employed in a next batch for carrying out the second stage.

10. Process according to claim 9, wherein filtrate is used again up to ten times.

11. Process according to claim 9, wherein only an organic phase, following removal of desired product, is re-used.
